(19) United States
(12) Patent Application Publication
(10) Pub. No.: US 2010/0125256 A1
(43) Pub. Date: May 20, 2010

(54) GLOVES AND SOCKS FOR SKIN CARE

(75) Inventor: Ildong KIM, Yongin-si (KR)

Correspondence Address: Jae Y. Park, Kile, Goekjian, Reed & McManus, PLLC, 1200 New Hampshire Ave. NW, Suite 570, Washington, DC, 20036, US

(73) Assignee: SKIN CARE KOREA CO., LTD, Yongin-si (KR)

(21) Appl. No.: 12/618,943

(22) Filed: Nov. 16, 2009

(30) Foreign Application Priority Data
Nov. 17, 2008 (KR) 20-2008-0015257

(51) Int. Cl. A61M 35/00

(52) U.S. Cl. 604/292; 604/293

(57) ABSTRACT

Provided are gloves and socks for skin care. The gloves for skin care include a first insertion part into which a hand is inserted and a second insertion part into which fingers are inserted. A first surface of a sheet forming the gloves is coated with cosmetic compositions, and forms an inner surface of the gloves that contacts the hand. A second surface, an opposite surface of the sheet is waterproofed, and forms an outer surface of the gloves that is exposed to the outside.

### CROSS-REFERENCE TO RELATED APPLICATIONS

This application claims priority under 35 U.S.C. §119 to Korean Utility Model Application No. 20-2008-0015257, filed on Nov. 17, 2008, in the Korean Intellectual Property Office, the disclosure of which is incorporated herein by reference in its entirety.

### TECHNICAL FIELD

The following disclosure relates to gloves and socks for skin care, and in particular, to gloves and socks for skin care, which have one surface of a sheet for forming the gloves and socks coated with a cosmetic composition and the other surface waterproofed, and can be manufactured using a single sheet.

### BACKGROUND

With the increase of attention to the skin care, skin care packs are widely being used for face and other body parts.

Typical skin care packs having gloves- or socks-shapes are manufactured by bonding a vinyl sheet forming an outer side of gloves or socks to a separate skin care sheet forming the inner side of the gloves or socks. However, when gloves and socks are manufactured by bonding the respective sheets, there are limitations in that the manufacturing process may be complex and a large amount of raw materials for the gloves and socks may be consumed.

Also, if the skin care sheet is attached to only one of upper and lower surfaces of the inner side of the gloves, there is an inconvenience in that it should be distinguished whether the glove is for the right hand or for the left hand before use.

### SUMMARY

Embodiments provide gloves and socks for skin care, which have one surface of a sheet for forming gloves and socks coated with cosmetic compositions and the other surface waterproofed, and thus do not require a separate sheet coated with cosmetic compositions, thereby simplifying the manufacturing process and reducing product price.

Embodiments also provide gloves and socks for skin care, which can be sealed along a cutting line to inhibit cosmetic compositions from being oxidized or flowing out before a user unseals the gloves and socks for skin care along the cutting line, and can be easily unsealed along the cutting line when a user intends to use the gloves and socks for skin care

Embodiments also provide gloves and socks for skin care, which can further include a friction part at the bottom of the gloves and socks and enables a user to grasp objects or walk with the gloves and socks put on, thereby realizing more convenient and safe skin care.

In one general aspect, gloves for skin care include a first insertion part into which a hand is inserted and a second insertion part into which fingers are inserted, wherein a first surface of a sheet forming the gloves is coated with cosmetic compositions, and forms an inner surface of the gloves that contacts the hand, and a second surface, an opposite surface of the sheet is waterproofed, and forms an outer surface of the gloves that is exposed to the outside.

The sheet may be a non-woven fabric.

The first insertion part may be sealed before use, have a cutting line formed thereon, and be unsealed along the cutting line to allow the hand to be inserted.

The first insertion part may include a sealing part sealing the first insertion part to inhibit the cosmetic compositions from contacting air before use.

The sealing part may include a coupling groove on an inner surface of the first insertion part and a coupling protrusion on another inner surface of the first insertion part, and the coupling protrusion may be engaged with the coupling groove.

The first insertion part may include a fixing part to inhibit the gloves from being taken off from the hand while a user puts on the gloves

The fixing part may be formed of one of a Velcro tape and a rubber band.

The second surface may include a friction part having protrusions at a position of fingers or a palm of the gloves to increase frictional force and inhibit the gloves from slipping.

In another general aspect, socks for skin care include a first insertion part into which a foot is inserted, wherein a first surface of a sheet forming the socks is coated with cosmetic compositions, and forms an inner surface of the socks that contacts the foot, and a second surface, an opposite surface of the sheet is waterproofed, and forms an outer surface of the socks that is exposed to the outside.

The first insertion part may be sealed before use, have a cutting line formed thereon, and be unsealed along the cutting line to allow the foot to be inserted.

The first insertion part may include a fixing part to inhibit the socks from being taken off from the foot while a user puts on the socks.

The fixing part may be formed of one of a Velcro tape and a rubber band.

The second surface may include a friction part having protrusions at a position of a sole of the foot to increase frictional force and inhibit the socks from slipping.

Other features and aspects will be apparent from the following detailed description, the drawings, and the claims.

### BRIEF DESCRIPTION OF THE DRAWINGS

FIG. 1 is a diagram illustrating gloves for skin care according to a first embodiment.

FIG. 2 is a diagram illustrating a sheet for forming gloves according to an embodiment.

FIG. 3 is a diagram illustrating a sheet including gloves patterns according to an embodiment.

FIG. 4 is a diagram taken along line A-A.

FIG. 5 is a diagram illustrating gloves for skin care including a cutting line according to an embodiment.

FIG. 6 is a diagram illustrating gloves for skin care including a sealing part according to an embodiment.

FIG. 7 is a diagram illustrating gloves for skin care including a fixing part according to an embodiment.

FIG. 8 is a diagram illustrating gloves for skin care including a fixing part according to an embodiment.

FIG. 9 is a diagram illustrating gloves for skin care including a friction part according to an embodiment.

FIG. 10 is a diagram illustrating socks for skin care according to a second embodiment.

FIG. 11 is a diagram illustrating socks for skin care including a cutting line according to an embodiment.

FIG. 12 is a diagram illustrating socks for skin care including a fixing part according to an embodiment.

FIG. 13 is a diagram illustrating socks for skin care including a fixing part according to an embodiment.

FIG. 14 is a diagram illustrating socks for skin care including a friction part according to an embodiment.

### DETAILED DESCRIPTION OF EMBODIMENTS

Hereinafter, exemplary embodiments will be described in detail with reference to the accompanying drawings. Throughout the drawings and the detailed description, unless otherwise described, the same drawing reference numerals will be understood to refer to the same elements, features, and structures. The relative size and depiction of these elements may be exaggerated for clarity, illustration, and convenience. The following detailed description is provided to assist the reader in gaining a comprehensive understanding of the methods, apparatuses, and/or systems described herein. Accordingly, various changes, modifications, and equivalents of the methods, apparatuses, and/or systems described herein will be suggested to those of ordinary skill in the art. Also, descriptions of well-known functions and constructions may be omitted for increased clarity and conciseness.

### First Embodiment

### Gloves for Skin Care

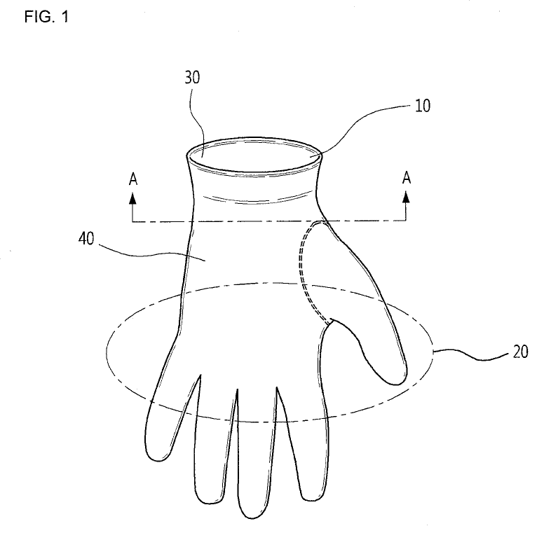

FIG. 1 is a diagram illustrating gloves for skin care according to a first embodiment.

Referring to FIG. 1, gloves for skin care according to an embodiment may include a first insertion part 10 into which a hand is inserted and a second insertion part 20 into which fingers are inserted. The second insertion part 20 into which fingers are inserted may have a five-finger shape. Alternatively, the gloves may be a mitten which covers the entire hand but do not have separate finger openings or sheaths.

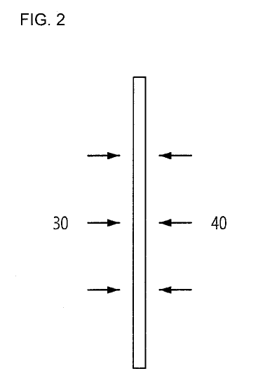

FIG. 2 is a diagram illustrating a sheet for forming gloves according to an embodiment.

Referring to FIG. 2, a sheet for forming gloves may have a first surface 30 and a second surface 40. The first surface 30 may be coated with cosmetic compositions, and may form the inner surface of the gloves when the gloves for skin care are manufactured. The second surface 40, the opposite surface of the first surface 30 may be waterproofed, and may form the outer surface of the gloves exposed to the outside when the gloves for skin care are manufactured. The waterproof process may include various methods such as attachment of a film or coating of liquid.

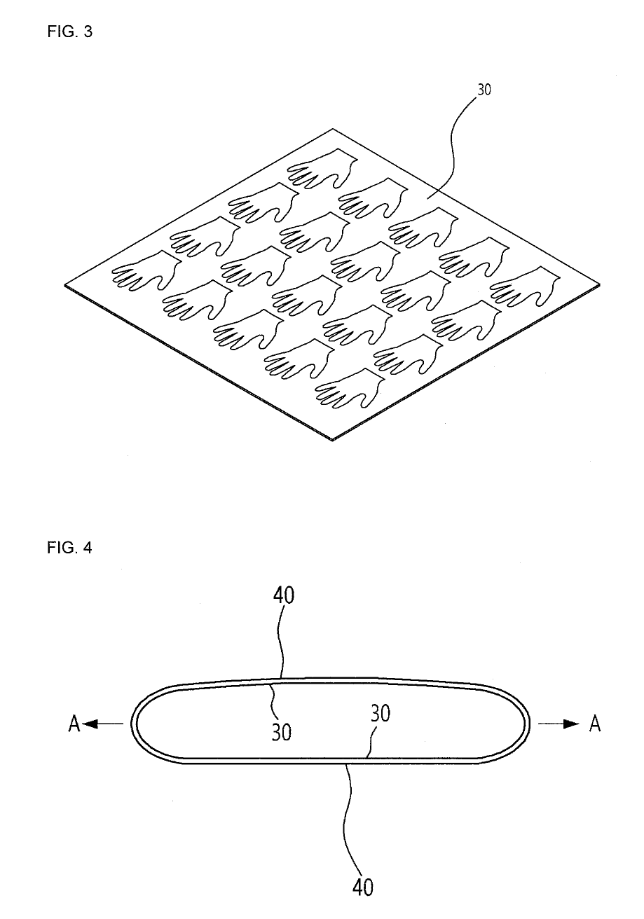

FIG. 3 is a diagram illustrating a sheet including gloves patterns according to an embodiment.

After the first surface of the sheet may be coated with cosmetic compositions, and the second surface of the sheet may be waterproofed, the sheet may be cut along the gloves pattern as shown in FIG. 3. Then, the gloves for skin care may be manufactured through a thermal bonding.

FIG. 4 is a diagram taken along line A-A. Referring to FIG. 4, the first surface 30 of the sheet may become the inner surface of the gloves, and the second surface 40 of the sheet may become the outer surface of the gloves. Accordingly, the inner surface of the gloves may be directly coated with cosmetic compositions, and the outer surface of the gloves may be waterproofed.

As illustrated in FIG. 4, since the upper and lower surfaces 30 of the inner surface of the gloves are both coated with cosmetic compositions, there is no inconvenience in that it is unnecessary to consider the right or left hand unlike related-art gloves having only one surface coated with cosmetic compositions

Also, since the first and second surfaces 30 and 40 are formed using a single sheet, a separate sheet coated with cosmetic compositions needs not to be attached to the inner side of the gloves. Accordingly, this embodiment provides effects of simplifying the manufacturing process and reducing the manufacturing cost.

The first surface 30 is coated with cosmetic compositions. The cosmetic compositions may contain various skin care ingredients such as creams, moisturizing ingredients, skin protection ingredients. When a user inserts his hands into the first insertion part 10, the cosmetic compositions may be applied over the skin of the hands. When a user puts on the gloves for hours time, the cosmetic compositions may be applied evenly over the hands, thereby providing an effect of improving the skin.

The second surface 40 may include a film to inhibit the cosmetic compositions coated on the first surface from flowing out of the gloves. Thus, the gloves do not need to be removed during the daily life. Also, since the gloves are sealed with a film, skin-moisturizing and nourishing effects may be maximized.

The sheet may be a non-woven fabric or other non-stimulating fabrics for the skin. The non-woven fabric has a higher compatibility with hands than vinyl used in a related-art, and thus provides an effect more consistent with the purposes of the present invention for increasing a skin care effect.

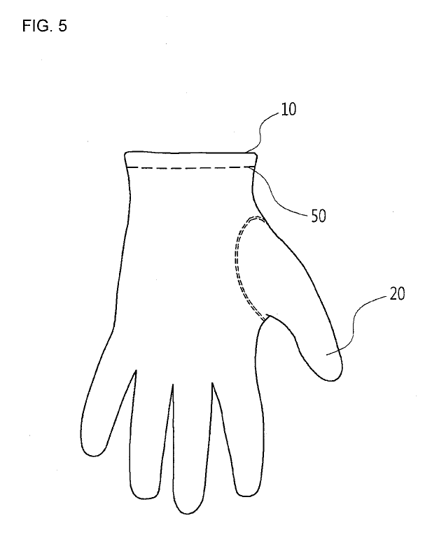

As illustrated in FIG. 5, the first insertion part 10 may be sealed when the gloves are manufactured. Also, a cutting line 50 is formed adjacent to the first insertion part. A user may unseal the gloves along the cutting line 50 and insert his hands into the gloves for use.

In gloves for skin care according to an embodiment, the first insertion part 10 may further include a sealing part 60. The sealing part 60 performs a function of sealing the first insertion part 10 to inhibit cosmetic compositions in the gloves for skin care from contacting air before use. The gloves may be sealed using the sealing part 60 before use, thereby inhibiting the cosmetic compositions from being oxidized or flowing out.

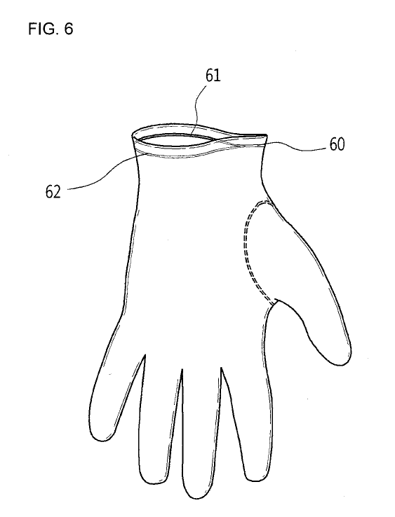

In this case, the sealing part 60 may be formed using a zipper clip as shown in FIG. 6. The zipper clip may include a coupling groove 61 formed on the inner surface at one side of the first insertion part 10 and a coupling protrusion 62 formed on the inner surface at the other side of the first insertion part 10 and engaged with the coupling groove 61.

Also, in gloves for skin care according to an embodiment, the first insertion part 10 may further include a fixing part. The fixing part serves to inhibit the gloves from being taken off when a user puts on the gloves for skin care.

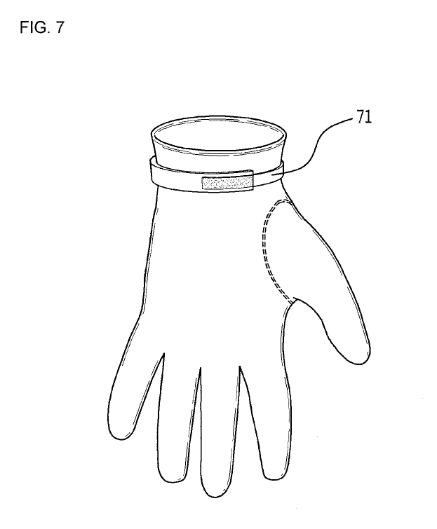

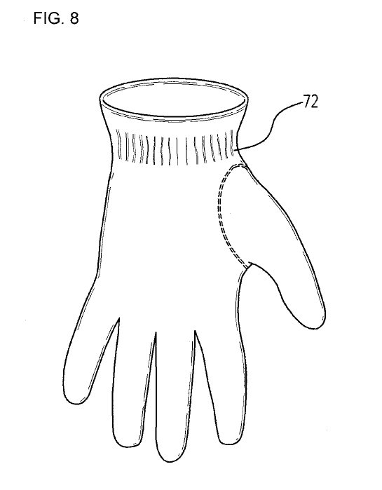

In this case, the fixing part may be formed using a Velcro tape as shown in FIG. 7, or a rubber band as shown in FIG. 8.

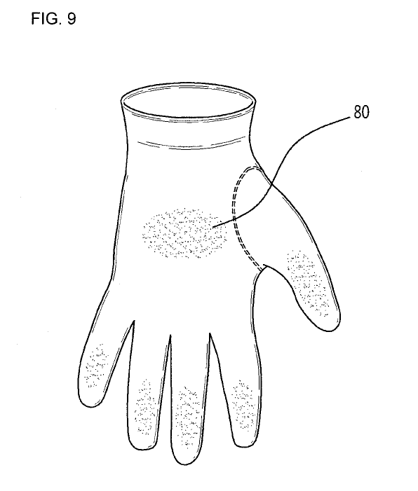

On the other hand, gloves for skin care according to an embodiment may further include a friction part 80 on the second surface 40. The friction part 80 may have protrusions at positions on the respective fingers and/or the palm as shown in FIG. 9, and may serve to inhibit slippage using a friction force. Thus, the embodiments enable a user to grasp objects without having to remove the gloves, thereby making daily life safer and more convenient.

### Second Embodiment

### Socks for Skin Care

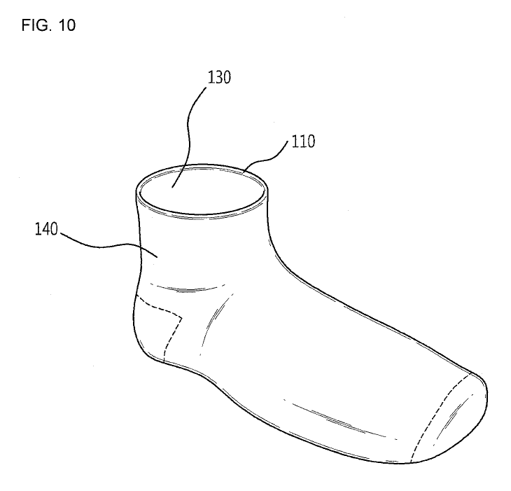

FIG. 10 is a diagram illustrating socks for skin care according to a second embodiment.

Socks for skin care may have a shape of typical socks including a first insertion part 110 into which the foot is inserted.

A sheet for forming socks may have a first surface 130 and a second surface 140. The first surface 130 may be coated with cosmetic compositions, and may form the inner surface of the socks that contacts the foot. The second surface 140, the opposite surface of the first surface 130 may be waterproofed, and may form the outer surface of the socks exposed to the outside.

Since the sheet for forming the socks is identical to the sheet for forming the gloves for skin care, detailed descriptions thereof will be omitted below.

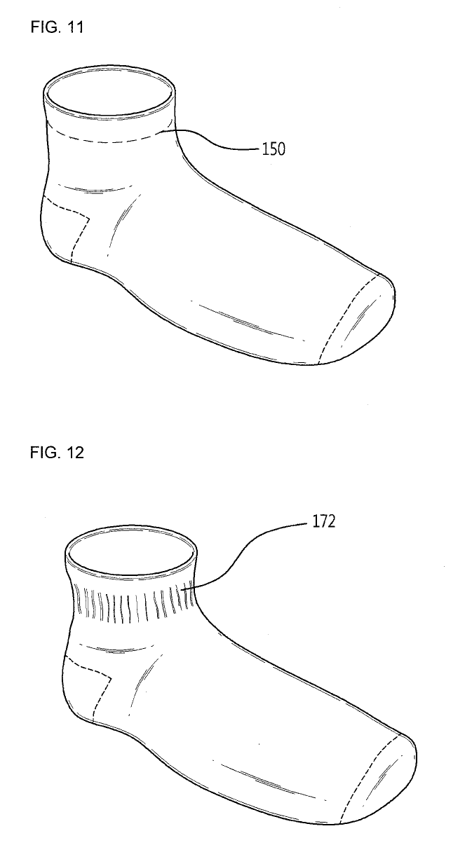

Socks for skin care according to an embodiment may have a cutting line 150 as shown in FIG. 11. The first insertion 110 may be sealed when being manufactured. In this case, a user may unseal the socks by releasing the cutting line 150.

Socks for skin care according to an embodiment may further include a fixing part on the first insertion part 110. The fixing part serves to inhibit the socks from being taken off from the foot.

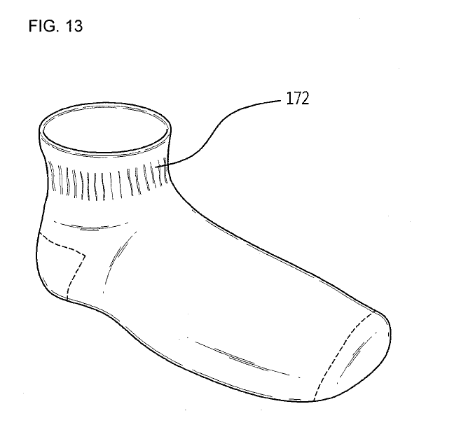

In this case, the fixing part may be formed using a Velcro tape as shown in FIG. 12 or a rubber band as shown in FIG. 13.

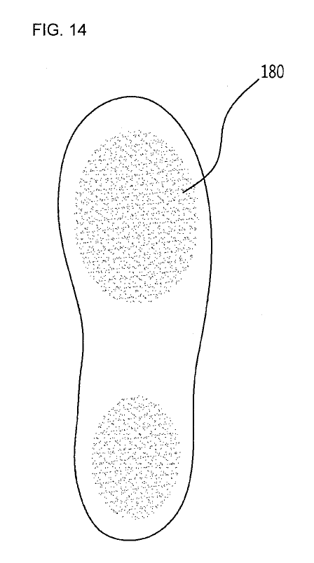

Socks for skin care according to an embodiment may further include a friction part 180 on the second surface 140. The friction part 180 may have protrusions at a position on the sole of the foot as shown in FIG. 14, and may serve to inhibit slippage using a friction force. Thus, the embodiments enable a user not to slide down without having to remove the socks, thereby making daily life safer and more convenient.

A number of exemplary embodiments have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. Gloves for skin care, comprising:
a first insertion part into which a hand is inserted; and
a second insertion part into which fingers are inserted,
wherein a first surface of a sheet forming the gloves is coated with cosmetic compositions, and forms an inner surface of the gloves that contacts the hand, and a second surface, an opposite surface of the sheet is waterproofed, and forms an outer surface of the gloves that is exposed to the outside.

2. The gloves for skin care of claim 1, wherein the sheet is a non-woven fabric.

3. The gloves for skin care of claim 1, wherein the first insertion part is sealed before use, has a cutting line formed thereon, and is unsealed along the cutting line to allow the hand to be inserted.

4. The gloves for skin care of claim 1, wherein the first insertion part comprises a sealing part sealing the first insertion part to inhibit the cosmetic compositions from contacting air before use.

5. The gloves for skin care of claim 4, wherein the sealing part comprises a coupling groove on an inner surface of the first insertion part and a coupling protrusion on another inner surface of the first insertion part, and the coupling protrusion is engaged with the coupling groove.

6. The gloves for skin care of claim 1, wherein the first insertion part comprises a fixing part to inhibit the gloves from being taken off from the hand while a user puts on the gloves.

7. The gloves for skin care of claim 6, wherein the fixing part is formed of one of a Velcro tape and a rubber band.

8. The gloves for skin care of claim 1, wherein the second surface comprises a friction part having protrusions at a position of fingers or a palm of the gloves to increase frictional force and inhibit the gloves from slipping.

9. Socks for skin care comprising: a first insertion part into which a foot is inserted,
wherein a first surface of a sheet forming the socks is coated with cosmetic compositions, and forms an inner surface of the socks that contacts the foot, and a second surface, an opposite surface of the sheet is waterproofed, and forms an outer surface of the socks that is exposed to the outside.

10. The socks for skin care of claim 9, wherein the first insertion part is sealed before use, has a cutting line formed thereon, and is unsealed along the cutting line to allow the foot to be inserted.

11. The socks for skin care of claim 9, wherein the first insertion part comprises a fixing part to inhibit the socks from being taken off from the foot while a user puts on the socks.

12. The socks for skin care of claim 11, wherein the fixing part is formed of one of a Velcro tape and a rubber band.

13. The socks for skin care of claim 9, wherein the second surface comprises a friction part having protrusions at a position of a sole of the foot to increase frictional force and inhibit the socks from slipping.
